(19) United States
(12) Patent Application Publication
(10) Pub. No.: US 2011/0071523 A1
(43) Pub. Date: Mar. 24, 2011

(54) Vessel Sealer with Self-Aligning Jaws

(75) Inventor: William J. Dickhans, Longmont, CO (US)

(73) Assignee: TYCO Healthcare Group LP

(21) Appl. No.: 12/565,281

(22) Filed: Sep. 23, 2009

(51) Int. Cl. A61B 18/18

(52) U.S. Cl. 606/45; 606/52

(57) ABSTRACT

An end effector assembly of a surgical forceps, the end effector assembly including first and second jaw members disposed in opposing relation relative to one another. At least one of the jaw members is moveable from an open position to a closed position for grasping tissue therebetween. The jaw members including opposing sealing surfaces configured to grasp tissue therebetween. Each of the opposing sealing surfaces has a knife channel defined therein. The opposing sealing surfaces of the first and second jaw members are shaped complementarily to one another to align the knife channels of the first and second jaw members upon movement from the first position to the second position.

### BACKGROUND

The present disclosure relates to a surgical forceps and, more particularly, the present disclosure relates to an electrosurgical forceps that includes self-aligning jaws.

### TECHNICAL FIELD

Electrosurgical forceps utilize both mechanical clamping action and electrical energy to affect hemostasis by heating tissue and blood vessels to coagulate, cauterize and/or seal tissue. As an alternative to open forceps for use with open surgical procedures, many modern surgeons use endoscopes and endoscopic instruments for remotely accessing organs through smaller, puncture-like incisions. As a direct result thereof, patients tend to benefit from less scarring and reduced healing time.

Endoscopic instruments are inserted into the patient through a cannula, or port, which has been made with a trocar. Typical sizes for cannulas range from three millimeters to twelve millimeters. Smaller cannulas are usually preferred, which, as can be appreciated, ultimately presents a design challenge to instrument manufacturers who must find ways to make endoscopic instruments that fit through the smaller cannulas.

Many endoscopic surgical procedures require cutting or ligating blood vessels or vascular tissue. Due to the inherent spatial considerations of the surgical cavity, surgeons often have difficulty suturing vessels or performing other traditional methods of controlling bleeding, e.g., clamping and/or tying-off transected blood vessels. By utilizing an endoscopic electrosurgical forceps, a surgeon can either cauterize, coagulate/desiccate and/or simply reduce or slow bleeding simply by controlling the intensity, frequency and duration of the electrosurgical energy applied through the jaw members to the tissue. Most small blood vessels, i.e., in the range below two millimeters in diameter, can often be closed using standard electrosurgical instruments and techniques. However, if a larger vessel is ligated, it may be necessary for the surgeon to convert the endoscopic procedure into an open-surgical procedure and thereby abandon the benefits of endoscopic surgery. Alternatively, the surgeon can seal the larger vessel or tissue. Typically, after a vessel or tissue is sealed, the surgeon advances a knife to sever the sealed tissue disposed between the opposing jaw members.

### SUMMARY

In accordance with the present disclosure, an end effector assembly of a surgical forceps is provided. The end effector assembly includes first and second jaw members disposed in opposing relation relative to one another that are moveable from an open position to a closed position for grasping tissue therebetween. The jaw members include opposing sealing surfaces configured to grasp tissue therebetween. Each of the jaw members also includes a knife channel defined therein. Opposing sealing surfaces of the first and second jaw members are shaped complementarily to one another to align the knife channels and sealing surfaces of the first and second jaw members upon movement from the first position to the second position.

In another embodiment, the opposing sealing surface of the first jaw member is concave and the opposing sealing surface of the second jaw member is complementarily convex. The concave opposing sealing surface may define a radial portion having a radius from a center point of the concavity and the convex opposing sealing surface may define a radial portion having a radius from a center point of the convexity wherein the radius of the concavity is substantially equal to the radius of the convexity.

In yet another embodiment, each opposing sealing surface angles inwardly from opposite longitudinal edges of the opposing sealing surface. The opposing sealing surfaces angle in the same direction with respect to a horizontal axis defined therethrough such that the jaw members are forced into alignment upon movement from the first position to the second position.

In another embodiment, the end effector assembly further includes one or more stop member disposed on the sealing surface of at least one of the jaw members.

A surgical forceps is also provided in accordance with the present disclosure that embodies a housing having at least one shaft attached thereto and an end effector assembly disposed at a distal end thereof. The end effector assembly includes first and second jaw members disposed in opposing relation relative to one another. One (or both) of the jaw members is moveable from an open position to a closed position for grasping tissue therebetween. The jaw members include opposing sealing surfaces configured to grasp tissue therebetween, each of the opposing sealing surfaces having a knife channel defined therein. A knife assembly is disposed within the shaft having a knife blade configured to translate distally from the shaft at least partially through the knife channels to cut tissue disposed between the jaw members. The opposing sealing surfaces of the first and second jaw members are shaped complementarily to one another to align the first and second jaws members upon movement from the first position to the second position.

In another embodiment, the surgical forceps includes at least one handle that moves the jaw members between the first and second positions.

In yet another embodiment, at least one of the jaw members is adapted to connect to an electrosurgical energy source to communicate energy to tissue disposed between the jaw members.

In still yet another embodiment in accordance with the present disclosure, a surgical forceps is provided. The forceps includes a housing having a shaft attached thereto. The shaft has an end effector assembly disposed at a distal end thereof. The end effector assembly includes first and second jaw members disposed in opposing relation relative to one another. One or both of the jaw members is moveable from an open position to a closed position for grasping tissue therebetween. The jaw members include opposing sealing surfaces configured to grasp tissue therebetween. The opposing sealing surface of the first jaw member is concave and the opposing sealing surface of the second jaw member is complementarily convex. The complementary-shaped sealing surfaces operate to align the first and second jaws members upon movement from the first position to the second position.

In another embodiment according to the present disclosure, a surgical forceps is provided. The forceps includes a housing having a shaft attached thereto. The shaft has an end effector assembly disposed at a distal end thereof. The end effector assembly includes first and second jaw members disposed in opposing relation relative to one another. One or both of the jaw members is moveable from an open position to a closed position for grasping tissue therebetween. The jaw members, which define a horizontal axis therethrough, include opposing sealing surfaces configured to grasp tissue therebetween. Each opposing sealing surface angles inwardly from opposite longitudinal edges of the opposing sealing surface in the same direction with respect to the horizontal axis such that the jaw members are forced into alignment upon movement from the first position to the second position.

### BRIEF DESCRIPTION OF THE DRAWINGS

Various embodiments of the subject instrument are described herein with reference to the drawings wherein:

FIG. 1 is a top, perspective view of an endoscopic forceps shown in an open configuration and including a housing, a handle assembly, a shaft and an end effector assembly for use with the present disclosure;

FIG. 2 is a top, perspective view of an alternate embodiment of an endoscopic forceps, including a housing, a handle assembly, a shaft and an end effector assembly;

FIG. 3 is a top, perspective view of an open surgical forceps, including a handle assembly, first and second shafts and an end effector assembly for use with the present disclosure;

FIG. 4 is an enlarged, side, perspective view of the end effector assembly of FIG. 1;

FIG. 5A is a front, cross-sectional view of the jaw members in an open configuration in accordance with one embodiment of the present disclosure;

FIG. 5B is a front, cross-sectional view of the jaw members of FIG. 5A, disposed in a closed configuration;

FIG. 6A is a front, cross-sectional view of the jaw members in an open configuration in accordance with another embodiment of the present disclosure;

FIG. 6B is a front, cross-sectional view of the jaw members of FIG. 6A, disposed in a closed configuration; and

FIG. 7 is a side, cross-sectional view of the end effector assembly showing a knife extending through the knife channels of the jaw members.

### DETAILED DESCRIPTION

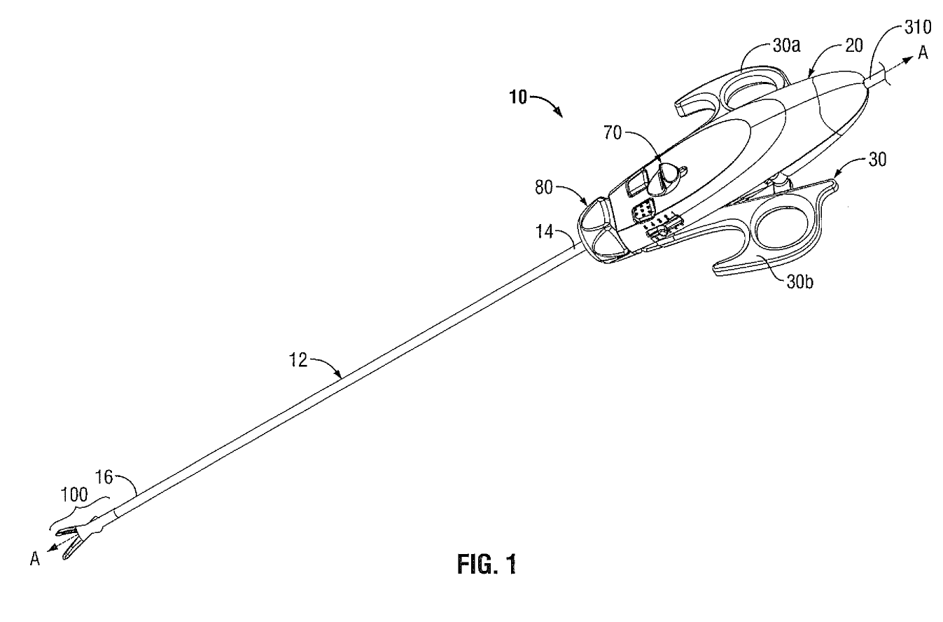

Turning now to FIG. 1, an endoscopic surgical forceps 10 is shown for use with various surgical procedures and generally includes a housing 20, a handle assembly 30, a rotating assembly 80, a knife trigger assembly 70 and an end effector assembly 100 which mutually cooperate to grasp, seal and divide tubular vessels and vascular tissue.

Forceps 10 includes a shaft 12 that has a distal end 16 dimensioned to mechanically engage the end effector assembly 100 and a proximal end 14 that mechanically engages the housing 20. The proximal end 14 of shaft 12 is received within the housing 20. In the drawings and in the descriptions which follow, the term “proximal”, as is traditional, will refer to the end of the forceps 10 which is closer to the user, while the term “distal” will refer to the end which is further from the user.

Forceps 10 also includes an electrosurgical cable 310 that connects the forceps 10 to a source of electrosurgical energy, e.g., a generator (not shown). Handle assembly 30 includes two movable handles 30a and 30b disposed on opposite sides of housing 20. Handles 30a and 30b are movable relative to one another to actuate the end effector assembly 100.

Rotating assembly 80 is mechanically coupled to housing 20 and is rotatable approximately 90 degrees in either direction about a longitudinal axis “A.” Rotating assembly 80, when rotated, rotates shaft 12, which, in turn, rotates end effector assembly 100. Such a configuration allows end effector assembly 100 to be rotated approximately 90 degrees in either direction with respect to housing 20. Details relating to the inner-working components of forces 10 are disclosed in commonly-owned U.S. patent application Ser. No. 11/540,335.

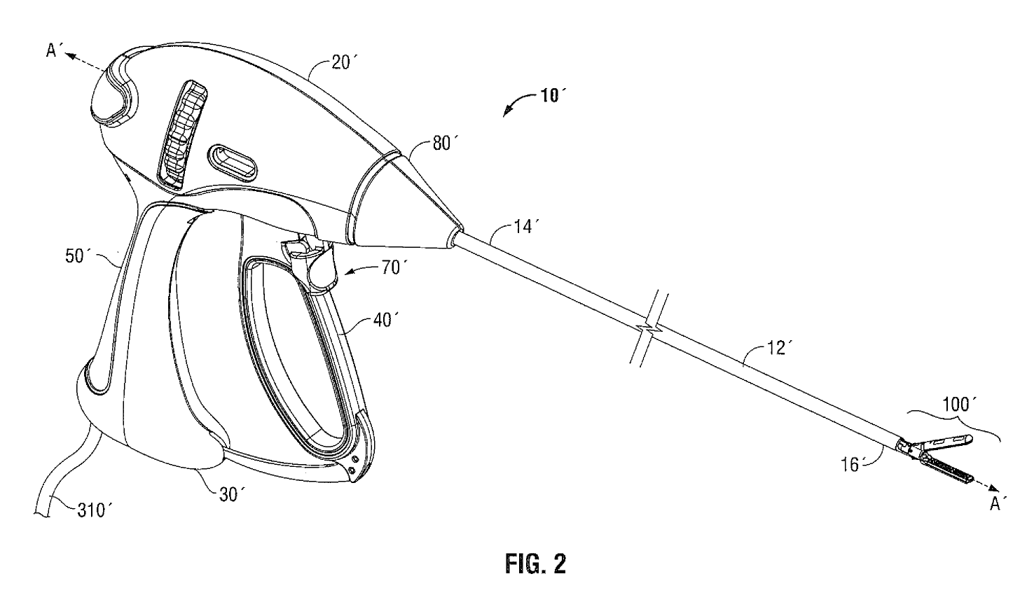

Referring now to FIG. 2, an alternate embodiment of an endoscopic forceps 10′ is shown that includes a housing 20′, a handle assembly 30′, a rotating assembly 80′, a trigger assembly 70′ and an end effector assembly 100′. Forceps 10′ further includes a shaft 12′ having a distal end 16′ configured to mechanically engage end effector assembly 100′ and a proximal end 16′ that mechanically engages housing 20′. Forceps 10′ also includes electrosurgical cable 310′ that connects forceps 10′ to a generator (not shown). Cable 310′ has sufficient length to extend through shaft 12′ in order to provide electrical energy to at least one of jaw members 110 and 120 of end effector assembly 100′.

With continued reference to FIG. 2, handle assembly 30′ includes fixed handle 50′ and a moveable handle 40′. Fixed handle 50′ is integrally associated with housing 20′ and handle 40′ is moveable relative to fixed handle 50′. Rotating assembly 80′ is integrally associated with housing 20′ and is rotatable approximately 180 degrees in either direction about a longitudinal axis “A′.” The housing 20′ includes two halves that house the internal working components of the forceps 10′.

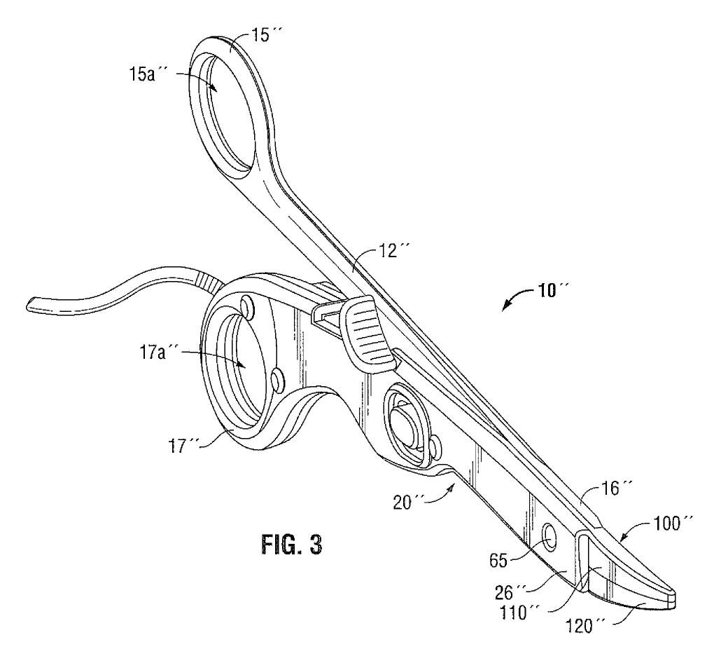

Referring now to FIG. 3, another alternate embodiment of a forceps 10″ for use with open surgical procedures is shown. Forceps 10″ includes end effector assembly 100″ that attaches to distal ends 16″ and 26″ of shafts 12″ and 20″, respectively. The end effector assembly 100″ includes a pair of opposing jaw members 110″ and 120″ which are pivotably connected about a pivot pin 65 and that are movable relative to one another to grasp tissue therebetween.

Each shaft 12″ and 20″ includes a handle 15″ and 17″, disposed at the proximal end thereof which each define a finger hole 15a″ and 17a″, respectively, therethrough for receiving a finger of the user. As can be appreciated, finger holes 15a″ and 17a″ facilitate movement of the shafts 12″ and 20″ relative to one another which, in turn, pivot the jaw members 110″ and 120″ from an open position wherein the jaw members 110″ and 120″ are disposed in spaced relation relative to one another to a clamping or closed position wherein the jaw members 110″ and 120″ cooperate to grasp tissue therebetween. End effector assembly 100″ is configured in a similar manner to the end effector assembly of FIGS. 1 and 2 above.

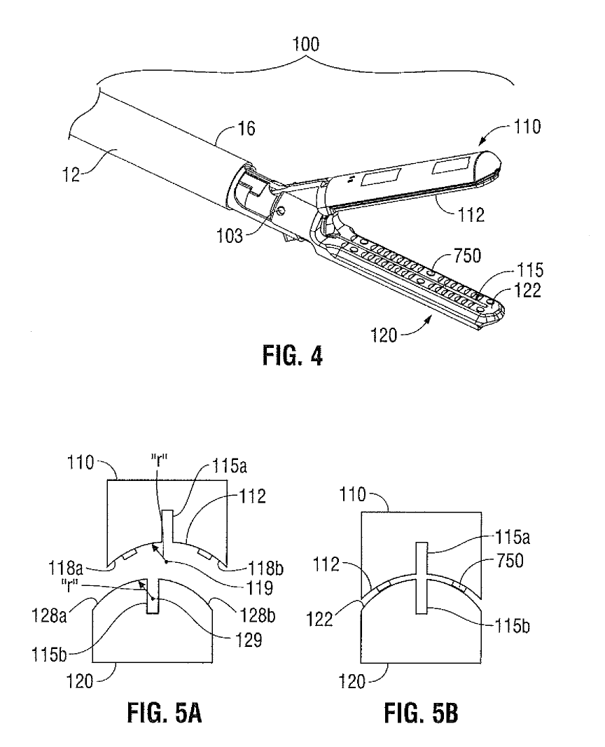

Referring now to FIG. 4, end effector assembly 100 is described with reference to the end effector assembly 100 show in FIG. 1. It is understood that all of the above end effector assemblies and forceps include similar designs and may be configured to accomplish the same purpose. End effector assembly 100 may be configured for mechanical attachment at the distal end 16 of shaft 12 of forceps 10. End effector assembly 100 includes a pair of opposing jaw members 110 and 120. Handles 30a and 30b of forceps 10 (see FIG. 1) ultimately connect to a respective drive assembly (not shown) which, together, mechanically cooperate to impart movement of the jaw members 110 and 120 from a first, open position wherein the jaw members 110 and 120 are disposed in spaced relation relative to one another, to a second, clamping or closed position wherein the jaw members 110 and 120 cooperate to grasp tissue therebetween. Details relating to the working components of the handle assembly and drive assembly of forceps 10 are disclosed in above-mentioned U.S. patent application Ser. No. 11/540,335.

With reference to the example embodiment of an end effector assembly 100 shown in FIG. 4, opposing jaw members 110 and 120 are pivotably connected about pivot 103. Jaw members 110 and 120 include electrically conductive sealing surfaces 112 and 122 that are dimensioned to securely engage tissue when clamped therebetween. A longitudinally-oriented knife channel 115 is defined between jaw members 110 and 120 for reciprocation of a knife 185 therethrough (see FIG. 7). Knife channel 115 is defined by channels 115a and 115b (see, e.g., FIGS. 5A-5B) disposed in the sealing surfaces 112 and 122, respectively. Alternatively, knife channel 115 may be defined completely within one of the sealing surfaces 112 and 122. Further, forceps 10 may be provided without the knife assembly (see FIG. 7) and, accordingly, the sealing surfaces 112 and 122 would be configured without the knife channel 115 defined therethrough. At least one of the jaw members 110, 120 includes an electrically insulative stop member (or members) 750 configured to control the gap distance between sealing surfaces 112 and 122 of jaw members 110 and 120, respectively.

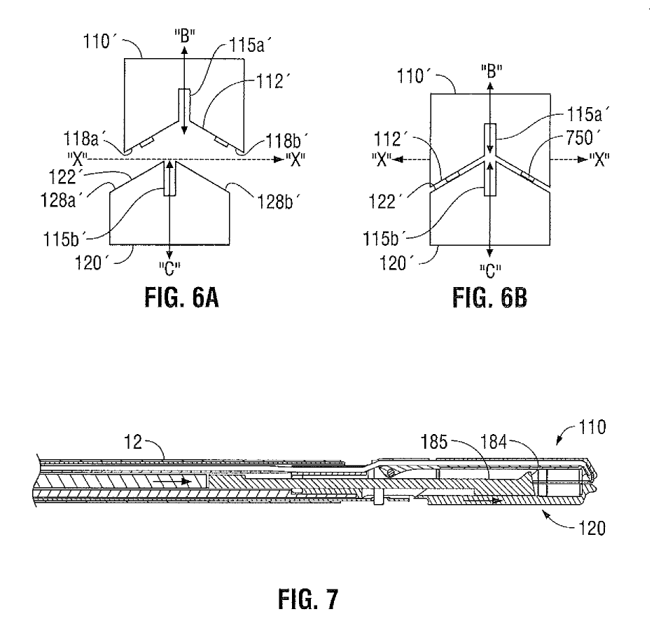

Features of jaw members 110 and 120 will now be described with reference to FIGS. 5A-5B and 6A-6B. FIG. 5A shows jaw members 110 and 120 disposed in a first, spaced-apart position. Sealing surface or opposing surface 112 of jaw member 110 has a generally concave shape. Sealing surface or opposing surface 122 of jaw member 120 has a generally convex shape. More specifically, sealing surface 112 defines an inward radial portion from opposite longitudinal sides 118a and 118b of sealing surface 112 having a radius “r” from a center point 119 of sealing surface 112. Opposing sealing surface 122 defines an outwardly protruding convex portion extending from opposite longitudinal sides 128a and 128b of sealing surface 122 and having a radius “r” which is substantially equal to the radius “r” of the radial portion defined within jaw member 110. Accordingly, opposing surface 112 and opposing surface 122 have complementary and, preferably non-linear shapes such that when the jaw members 110 and 120 are moved into the second, or closed position, the concave radial portion of jaw member 110 and the convex radial portion of jaw member 120 fit together, as shown in FIG. 5B.

These complementary-shaped opposing surfaces 112 and 122 of FIGS. 5A-5B align the jaw members 110 and 120 as described hereinbelow. For example, as shown in FIG. 5A, due to the inherent splay which results when two surfaces connected about a pivot come together, jaw members 110 and 120 may be offset from one another as the jaw members 110 and 120 move to and from open and closed positions. For example, as shown in FIG. 5A, jaw member 110 is offset relative to jaw member 120. As jaw members 110 and 120 move to the position shown in FIG. 5B, jaw member 110 is forced into alignment with jaw member 120, so that the complementary opposing surfaces 112 and 122 fit together.

Further, the self-aligning feature of the above-described complementary-shaped opposing surfaces 112 and 122 ensures alignment of knife channels 115a and 115b as jaw members 110 and 120 move from an open to a closed position. The alignment of knife channels 115a and 115b, as shown in FIG. 5B, allows knife blade 184 of knife 185 (see FIG. 7) to more easily translate through knife channel 115 to cut tissue disposed between jaw members 110 and 120. Additionally, the complementary concave and convex sealing surfaces 112 and 122, respectively, provide a larger seal width as compared to linear sealing surfaces having the same overall width. On the other hand, the complementary concave and convex sealing surfaces 112 and 122, respectively, allow jaw members 110 and 120 to be constructed with an overall smaller width, while maintaining an equal seal width as compared to jaw members having linear sealing surfaces.

Referring now to FIGS. 6A-B, another embodiment of the present disclosure is shown wherein opposing surface 112′ of jaw member 110′ is angled inwardly from opposite longitudinal sides 118a′ and 118b′ of sealing surface 112′ toward centerline “B” of jaw member 110′. Opposing surface 122′ of jaw member 120′ is angled inwardly from opposite longitudinal sides 128a′ and 128b′ to centerline “C” of jaw member 120′. Opposing surfaces 112′ and 122′ are angled in the same direction with respect to horizontal axis “X” and thus, as in the embodiment of FIGS. 5A-5B, opposing surfaces 112′ and 122′ of FIGS. 6A-6B are complementarily-shaped relative to one another. Thus, as jaw members 110′ and 120′ move from the first position to the second position, the jaw members 110′ and 120′ are forced into alignment as a result of their respective complementary shapes. Likewise, the alignment of knife channels 115a′ and 115b′ is aided by the shape of opposing surface 112′ and complementary opposing surface 122′.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly of a surgical forceps, the end effector assembly comprising:
first and second jaw members disposed in opposing relation relative to one another, at least one of the jaw members moveable from an open position to a closed position for grasping tissue therebetween, the jaw members including opposing sealing surfaces configured to grasp tissue therebetween, each of the opposing sealing surfaces having a knife channel defined therein;
wherein the opposing sealing surfaces of the first and second jaw members are shaped complementarily to one another to align the knife channels and sealing surfaces of the first and second jaw members upon movement from the first position to the second position.

2. The end effector assembly according to claim 1, wherein the opposing sealing surface of the first jaw member is concave and wherein the opposing sealing surface of the second jaw member is complementarily convex.

3. The end effector assembly according to claim 2, wherein the concave opposing sealing surface defines a radial portion having a radius from a center point of the concavity and wherein the convex opposing sealing surface defines a radial portion having a radius from a center point of the convexity, wherein the radius of the concavity is substantially equal to the radius of the opposing convexity.

4. The end effector assembly according to claim 1, wherein a horizontal axis is defined through the jaw members and wherein each opposing sealing surface angles inwardly from opposite longitudinal edges of the opposing sealing surface in the same direction with respect to the horizontal axis such that the jaw members are forced into alignment upon movement from the first position to the second position.

5. The end effector assembly according to claim 1, further comprising at least one stop member disposed on the sealing surface of at least one of the first and second jaw members.

6. A surgical forceps, the forceps comprising:
a housing having at least one shaft attached thereto, the shaft having an end effector assembly disposed at a distal end thereof, wherein the end effector assembly includes:
first and second jaw members disposed in opposing relation relative to one another, at least one of the jaw members moveable from an open position to a closed position for grasping tissue therebetween, the jaw members including opposing sealing surfaces configured to grasp tissue therebetween, each of the opposing sealing surfaces having a knife channel defined therein; and
a knife assembly disposed within the shaft, the knife assembly having a knife blade configured to translate distally from the shaft at least partially through the knife channels to cut tissue disposed between the jaw members;
wherein opposing sealing surfaces of the first and second jaw members are shaped complementarily to one another to align the knife channels of the first and second jaws members upon movement from the first position to the second position.

7. The surgical forceps according to claim 6, wherein the forceps further includes at least one handle that moves the jaw members between the first and second positions.

8. The surgical forceps according to claim 6, wherein at least one of the jaw members is adapted to connect to an electrosurgical energy source to communicate energy to tissue disposed between the jaw members.

9. A surgical forceps, the forceps comprising:
a housing having a shaft attached thereto, the shaft having an end effector assembly disposed at a distal end thereof, wherein the end effector assembly includes:
first and second jaw members disposed in opposing relation relative to one another, at least one of the jaw members moveable from an open position to a closed position for grasping tissue therebetween, the jaw members including opposing sealing surfaces configured to grasp tissue therebetween, wherein the opposing sealing surface of the first jaw member is concave and wherein the opposing sealing surface of the second jaw member is complementarily convex such that the complementary-shaped sealing surfaces operate to align the first and second jaws members upon movement from the first position to the second position.

10. A surgical forceps, the forceps comprising:
a housing having a shaft attached thereto, the shaft having an end effector assembly disposed at a distal end thereof, wherein the end effector assembly includes:
first and second jaw members disposed in opposing relation relative to one another, at least one of the jaw members moveable from an open position to a closed position for grasping tissue therebetween, the jaw members including opposing sealing surfaces configured to grasp tissue therebetween, wherein a horizontal axis is defined through the jaw members and wherein each opposing sealing surface angles inwardly from opposite longitudinal edges of the opposing sealing surface in the same direction with respect to the horizontal axis such that the jaw members are forced into alignment upon movement from the first position to the second position.
